(19) United States
(12) Patent Application Publication
(10) Pub. No.: US 2017/0128658 A1
(43) Pub. Date: May 11, 2017

(54) ARTERIOVENOUS ACCESS VALVE SYSTEM WITH SEPARATE VALVE TUBES

(71) Applicant: CreatiVasc Medical Inc., Greenville, SC (US)

(72) Inventors: James S. Johnson, Greenville, SC (US); Frank Patterson, Taylors, SC (US); David L. Cull, Greenville, SC (US)

(73) Assignee: CreatiVasc Medical Inc.

(21) Appl. No.: 14/938,003

(22) Filed: Nov. 11, 2015

(51) Int. Cl. A61M 1/36; A61M 1/16; A61M 39/10; A61F 2/06; A61M 39/02; A61M 39/22

(52) U.S. Cl. CPC A61M 1/3655; A61M 39/0208; A61M 39/227; A61M 39/10; A61F 2/06; A61M 1/16

(57) ABSTRACT

An arteriovenous access valve system may generally include a first valve tube extending lengthwise between an arterial end and a first joint end, with the first valve tube including a first valve device. The system may also include a separate second valve tube extending lengthwise between a venous end and a second joint end, with the second valve tube including a second valve device. In addition, the system may include at least one tube connector coupled between the first and second valve tubes.

### FIELD OF THE INVENTION

The present subject matter relates generally to arteriovenous access valve systems and, more particularly, to an arteriovenous access valve system including separate valve tubes and one or more tube connectors configured to couple the valve tubes together.

### BACKGROUND OF THE INVENTION

The function of kidneys, which are glandular organs located in the upper abdominal cavity of vertebrates, is to filter blood and remove waste products. Specifically, kidneys separate water and waste products of metabolism from blood and excrete them as urine through the bladder. Chronic renal failure is a disease of the kidney in which the kidney function breaks down and is no longer able to filter blood and remove waste substances. Should certain toxic waste substances not be removed from the blood, the toxic substances may increase to lethal concentrations within the body.

Hemodialysis is a life-sustaining treatment for patients who have renal failure. Hemodialysis is a process whereby the patient's blood is filtered and toxins are removed using an extracorporeal dialysis machine. For hemodialysis to be effective, large volumes of blood must be removed rapidly from the patient's body, passed through the dialysis machine, and returned to the patient. A number of operations have been developed to provide access to the circulation system of a patient such that patients may be connected to the dialysis machine.

For example, the most commonly performed hemodialysis access operation is a subcutaneous placement of an arteriovenous graft, which is made from a biocompatible tube. The biocompatible tube can be made of, for instance, a fluoropolymer such as polytetrafluoroethylene. One end of the tube is connected to an artery while the other end is connected to a vein. The arteriovenous graft is typically placed either in the leg or arm of a patient.

Blood flows from the artery, through the graft and into the vein. To connect the patient to a dialysis machine, two large hypodermic needles are inserted through the skin and into the graft. Blood is removed from the patient through one needle, circulated through the dialysis machine, and returned to the patient through the second needle. Typically, patients undergo hemodialysis approximately four hours a day, three days a week.

Various problems, however, have been experienced with the use of an arteriovenous graft. For example, arterial steal occurs when excessive blood flow through the arteriovenous graft “steals” blood from the distal arterial bed. Arterial steal can prevent the proper supply of blood from reaching the extremities of a patient.

To address such problems, systems and processes have been deployed which can minimize or prevent complications by closing the arteriovenous graft when hemodialysis is not taking place. An example of one such system is described in U.S. Pat. No. 7,025,741 entitled “Arteriovenous Access Valve System and Process”, which is hereby incorporated by reference herein in its entirety for all purposes. These systems and processes utilize two separate valve assemblies, such as balloon valve assemblies, to force closure of an arteriovenous graft by pressing the opposite ends of the arteriovenous graft walls together.

However, such valve systems currently require that the two valve assemblies be surgically connected to a conventional arteriovenous graft via end-to-end anastomoses after such valve assemblies have already been anastomosed to the artery and the vein of the patient. As a result, current valve systems require a total of four anastomoses (e.g., around 60-80 stiches), which significantly impacts the overall length of the surgery required to implant such conventional valve systems. Moreover, the requirement of four separate anastomoses can make the corresponding surgery quite tedious

Accordingly, an improved valve system that reduces the surgery time and/or tedious nature of implanting the system within a patient would be welcomed in the technology.

### BRIEF DESCRIPTION OF THE INVENTION

Aspects and advantages of the invention will be set forth in part in the following description, or may be obvious from the description, or may be learned through practice of the invention.

In one aspect, the present subject matter is directed to an arteriovenous access valve system. The system may generally include a first valve tube extending lengthwise between an arterial end and a first joint end, with the first valve tube including a first valve device. The system may also include a separate second valve tube extending lengthwise between a venous end and a second joint end, with the second valve tube including a second valve device. In addition, the system may include at least one tube connector coupled between the first and second valve tubes.

In another aspect, the present subject matter is directed to an arteriovenous access valve system. The system may generally include a first valve tube extending lengthwise between an arterial end and a first joint end, with the first valve tube including a first valve device and a first tube portion extending outwardly from the first valve device towards the first joint end. The system may also include a separate second valve tube extending lengthwise between a venous end and a second joint end, with the second valve tube including a second valve device and a second tube portion extending outwardly from the second valve device towards the second joint end. In addition, the system may include a tube connector extending lengthwise between a first end and a second end. The first joint end of the first valve tube may be configured to be coupled to the tube connector at or adjacent to the first end and the second joint end of the second valve tube may be configured to be coupled to the tube connector at or adjacent to the second end.

In a further aspect, the present subject matter is directed to an arteriovenous access valve system. The system may generally include a first valve tube extending lengthwise between an arterial end and a first joint end, with the first valve tube including a first valve device. The system may also include a separate second valve tube extending lengthwise between a venous end and a second joint end, with the second valve tube including a second valve device. In addition, the system may include a graft and first and second tube connectors, with the graft extending lengthwise between a first graft end and a second graft end. The first tube connector may be configured to couple the first joint end of the first valve tube to the first graft end of the graft and the second tube connector may be configured to couple the second end of the graft to the second joint end of the second valve tube.

In yet another aspect, the present subject matter is directed to a method for providing access between an artery and a vein of a patient. The method may generally include subcutaneously implanting an arteriovenous access valve system in the patient. The arteriovenous access valve system may include a first valve tube extending lengthwise between an arterial end and a first joint end and a separate second valve tube extending lengthwise between a venous end and a second joint end. The first valve tube may include a first valve device and the second valve tube may include a second valve device. The arteriovenous access valve system may also include at least one tube connector configured to be coupled between the first and second valve tubes. In addition, the method may include opening the first and second valve devices to allow blood to flow through the first and second valve tubes between the artery and the vein of the patient and closing the first and second valve devices to prevent blood from flowing through the first and second valve tubes between the artery and the vein of the patient.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a side view with cut away portions of a human arm illustrating one example of the placement of a conventional arteriovenous graft;

FIG. 2 illustrates a simplified, perspective view of one embodiment of an arteriovenous access valve system in accordance with aspects of the present subject matter;

FIG. 3 illustrates a perspective, assembled view of various components of the arteriovenous access valve system shown in FIG. 2 in accordance with aspects of the present subject matter, particularly illustrating first and second valve tubes of the system coupled to one another via a tube connector;

FIG. 4 illustrates a perspective, unassembled view of the components shown in FIG. 3;

FIG. 5 illustrates a close-up, assembled view of portions of the components shown in FIG. 3, particularly illustrating the connection between the separate valve tubes and the tube connector;

FIG. 6 illustrates a perspective view of the tube connector shown in FIGS. 3-5;

FIG. 7 illustrates a cross-sectional view of one embodiment of a valve device suitable for use within the disclosed system in accordance with aspects of the preset subject matter, particularly illustrating the valve device in a closed position;

FIG. 8 illustrates another cross-sectional view of the valve device shown in FIG. 7, particularly illustrating the valve device in an opened position;

FIG. 9 illustrates another perspective, unassembled view of the components shown in FIG. 3, particularly illustrating end portions of the first and second valve tubes being cut-off to adjust the length of the valve tubes;

FIG. 10 illustrates a perspective, assembled view of the components shown in FIG. 9, particularly illustrating the shortened valve tubes coupled to the tube connector;

FIG. 11 illustrates an assembled view of various components that may be included within another embodiment of an arteriovenous access valve system in accordance with aspects of the present subject matter;

FIG. 12 illustrates an unassembled view of the various system components shown in FIG. 11; and

FIG. 13 illustrates a cross-sectional view of a portion of the system components shown in FIG. 11 taken about line 13-13.

### DETAILED DESCRIPTION OF THE INVENTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, the present subject matter is directed to an arteriovenous access valve system including separate valve tubes configured to be coupled together to form a continuous flow path between an artery and a vein of a patient. Specifically, in several embodiments, the system may include a first valve tube extending between an arterial end and a first joint end, with the arterial end being configured to be coupled to the patient's artery. In addition, the system may include a separate, second valve tube extending between a venous end and a second joint end, with the venous end configured to be coupled to the patient's vein. Each valve tube may also include a valve device integrated therein or otherwise associated therewith. For instance, the first valve tube may include a first valve device disposed at or adjacent to its arterial end. Similarly, the second valve tube may include a second valve device disposed at or adjacent to its venous end. The first and second valve devices may generally be configured to be actuated between an opened position and a closed position to allow or prevent blood flow through the valve tubes, respectively.

Moreover, the system may also include one or more tube connectors configured to couple the separate valve tubes together. For instance, as will be described below, in one embodiment, the system may include a single tube connector extending lengthwise between a first end and a second end, with the first end being configured to be coupled to the first joint end of the first valve tube (e.g., via one or more loop stiches) and the second end being configured to be coupled to the second joint end of the second valve tube (e.g., via one or more loop stiches). As a result, the tube connector may serve as a direct fluid coupling between the first and second valve tubes to allow a continuous flow path to be defined between the patient's artery and vein. In another embodiment, the system may include first and second tube connectors configured to couple the separate vale tubes together via a graft. For instance, the first tube connector may be coupled between the first valve tube and one end of the graft and the second tube connector may be coupled between the second valve tube and the opposed end of the graft. As such, the tube connectors may serve as fluid couplings between the first and second valve tubes and the graft to allow a continuous flow path to be defined between the patient's artery and vein.

It should be appreciated that the disclosed arteriovenous access valve system may generally provide numerous advantages over conventional systems. For example, as opposed to the four anastomoses required by traditional systems, the disclosed system only requires two anastomoses, one to couple the arterial end of the first valve tube to the patient's artery and another to couple the venous end of the second valve tube to the patient's vein. Such a reduction in the number of anastomoses significantly reduces the amount of surgery time required to implant the components of the disclosed system into a patient as well as reduces the overall complexity of the surgical procedure.

In addition, the disclosed system components may be adjustable to meet the needs of the patient. Specifically, in embodiments in which the separate valve tubes are configured to be coupled together via a common connector, the length of each valve tube may be adjusted, as is necessary or desired, prior to connecting the tubes to one another. For example, each valve tube may be cut to the desired length required to suit the specific patient's body size, vascular system, access type and/or access location (e.g., the patient's arm versus the patient's thigh). The cut ends of the valve tubes may then be attached to the tube connector to fluidly couple the first valve tube to the second valve tube. Similarly, in embodiments in which the valve tubes are configured to be coupled together via separate tube connectors and a graft, the length of each valve tube and/or the length of the graft may be adjusted, as is necessary or desired, to customize the system components to the patient.

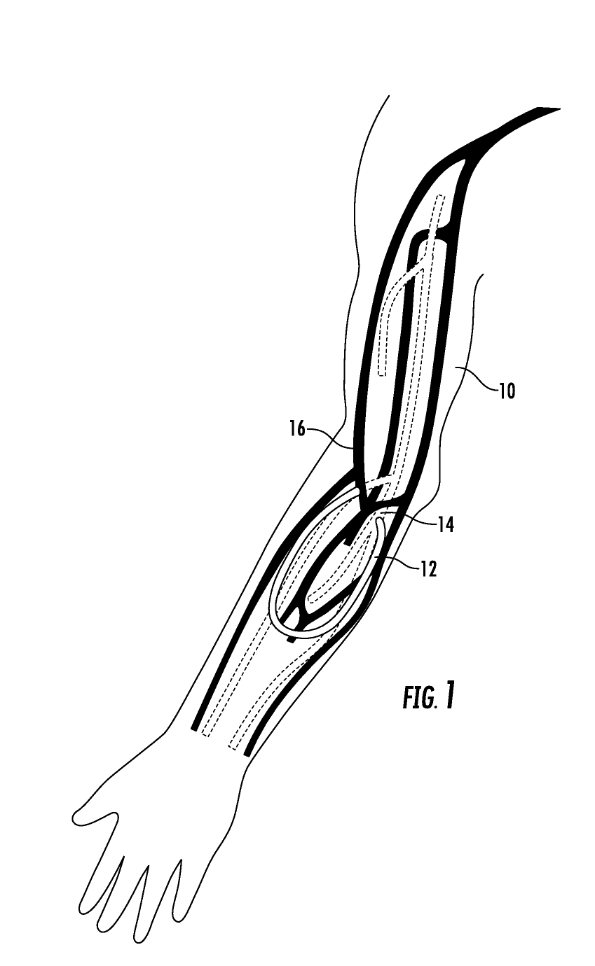

Referring now to FIG. 1, for purposes of explanation, a right arm 10 of a patient is shown. Selected arteries (shown as dotted pathways) are illustrated in conjunction with selected veins (shown as dark pathways). An arteriovenous graft 12 is shown connected at one end to an artery 14 of the patient (e.g., a brachial artery) and at an opposite end to a vein 16 of the patent (e.g., the cephalic vein).

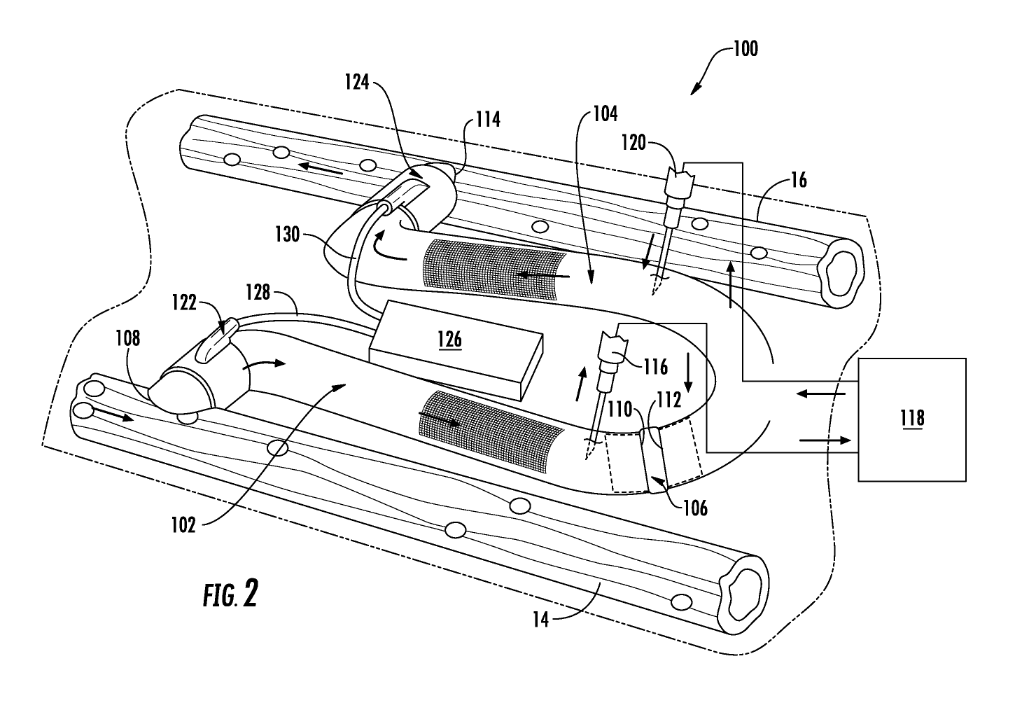

Referring now to FIG. 2, one embodiment of an arteriovenous access valve system 100 is illustrated in accordance with aspects of the present subject matter. As shown, the system 100 may include a first valve tube 102 and a second valve tube 104 coupled together via a tube connector 106 such that the first and second valve tubes 102, 104 generally extend between an artery 14 and a vein 16 of a patient. In general, the first valve tube 102 may be configured to extend between an arterial end 108 configured to be coupled to the patient's artery 14 (e.g., via an anastomoses) and a first joint end 110 configured to be coupled to the tube connector 106. Similarly, the second valve tube 104 may be configured to extend between a second joint end 112 configured to be coupled to the tube connector 106 and a venous end 114 configured to be coupled to the patient's vein 16. As such, when coupled together via the tube connector 106, the first and second valve tubes 102, 104 may generally define a continuous flow path for blood extending between the artery 14 and the vein 16.

In order to carry out hemodialysis, a first hypodermic needle 116 may be inserted through the skin and into either the first valve tube 102 or the second valve tube 104. Blood is removed from the flowpath formed by the first and second valve tubes 102, 104 through the needle 116 and into a dialysis machine 118. In the dialysis machine 118, waste materials are removed from the blood. After circulating through the dialysis machine 118, the blood is then fed back into the flowpath defined by the first and second valve tubes 102, 104 via a second hypodermic needle 120 at a location downstream of the first hypodermic needle 116.

In the illustrated embodiment, the first hypodermic needle 116 is shown as being inserted into the first valve tube 102 and the second hypodermic needle 120 is shown as being inserted into the second valve tube 104. However, in other embodiments, the first and second hypodermic needles 116, 120 may be both inserted into the first valve tube 102 or the second valve tube 104.

In addition, the first and second valve tubes 102, 104 may each include a valve device 122, 124 integrated therein and/or otherwise associated therewith. For example, as shown in FIG. 2, the first valve tube 102 may include a first valve device 122 positioned at or adjacent to its arterial end 108 and the second valve tube 104 may include a second valve device 124 positioned at or adjacent to its venous end 114. The valve devices 122, 124 may be configured to be in an opened position during normal hemodialysis (e.g., as shown in FIG. 2). When hemodialysis has ended, however, the valve devices 122, 124 may be moved to a closed position in order to prevent blood flow through the valve tubes 102, 104. In this manner, arterial steal is either eliminated or reduced.

In several embodiments, the valve devices 122, 124 may correspond to balloon-actuated valves and, thus, may each include an inflatable balloon (not shown in FIG. 2). When inflated, the balloons close the valve devices 122, 124 in a manner that reduces or eliminates the blood flow through the valve tubes 102, 104. In contrast, when the balloons are deflated, the valve devices 122, 124 are opened and blood may be directed along the flow path defined by the valve tubes 102, 104 between the patient's artery 14 and vein 16. To provide for such inflation/deflation of the balloons, the system 100 may also include an actuator assembly 126 in fluid communication with the first and second valve devices 122, 124. For example, as shown in the illustrated embodiment, the actuator assembly 126 may be in fluid communication with the first valve device 122 via tubing 128 and may be in fluid communication with the second valve device 124 via tubing 130. Thus, in embodiments in which the valve devices 122, 124 correspond to balloon-actuated valves, the actuator assembly 126 may be configured to supply a suitable fluid (e.g., saline) to the first and second valve device 122, 124 via the corresponding tubing 128, 130 to inflate the balloons, thereby closing the valve devices 122, 124. Similarly, when it is desired to open the valve devices 122, 124, the actuator assembly 126 may be configured to draw the fluid out of the balloons, thereby deflating the balloons and allowing blood to flow through the first and second valve tubes 102, 104.

It should be appreciated that, the actuator assembly 126 may generally correspond to any suitable device(s) or component(s) that is configured to actuate or adjust the valve devices 122, 124 between their opened and closed positions. For instance, in one embodiment, the actuator assembly 126 may correspond to a magnetically activated actuator assembly. One example of a suitable magnetically activated actuator assembly is described in U.S. patent application Ser. No. 14/695,241 (Johnson et al.), filed on Apr. 24, 2015 and entitled “Magnetically Activated Arteriovenous Access Valve System and Related Methods,” the disclosure of which is hereby incorporated by reference herein in its entirety for all purposes. For instance, the actuator assembly 126 may include an implanted housing configured to house both a driver assembly (e.g., a gear pump or a plunger device) and a drive magnet that is rotatably coupled to the driver assembly. In such an embodiment, rotation of the drive magnet may rotatably drive the driver assembly so as to supply fluid to the first and second valve devices 122, 124 or to draw fluid out of the first and second valve devices 122, 124 depending on a rotational direction of the driver assembly. As described in the '241 application, such rotation of the drive magnet may be achieved, for example, using an external activator device including a rotatable activator magnet. Thus, by placing the external activator device adjacent to the exterior of the patient's skin, rotation of the activator magnet may rotate the drive magnet, thereby driving the driver assembly and causing the valve devices 122, 124 to be opened or closed.

In another embodiment, the actuator assembly 126 may correspond to an implanted device configured to receive the tip of a hypodermic needle to allow fluid to be supplied to and drawn out of the valve devices 122, 125. One example of such an actuator assembly is described in U.S. Pat. No. 8,764,698 (Cull) entitled “Arteriovenous Access Valve System and Process,” the disclosure of which is hereby incorporated by reference herein in its entirety for all purposes. For instance, the actuator assembly 126 may include an implanted housing defining a septum or injection port for receiving the tip a hypodermic needle. In such an embodiment, when fluid is injected or withdrawn through the septum or injection port via the needle, the valve devices 122, 124 may be opened or closed, respectively.

In alternative embodiments, it should be appreciated that the actuator assembly 126 may correspond to any other suitable device(s) or component(s) that is configured to actuate or adjust the valve devices 122, 124 between their opened and closed positions. For instance, any device(s) or component(s) that allows fluid to be supplied to and/or drawn out of the valve devices 122, 125 may correspond to an actuator assembly 126 suitable for use in the disclosed system 100.

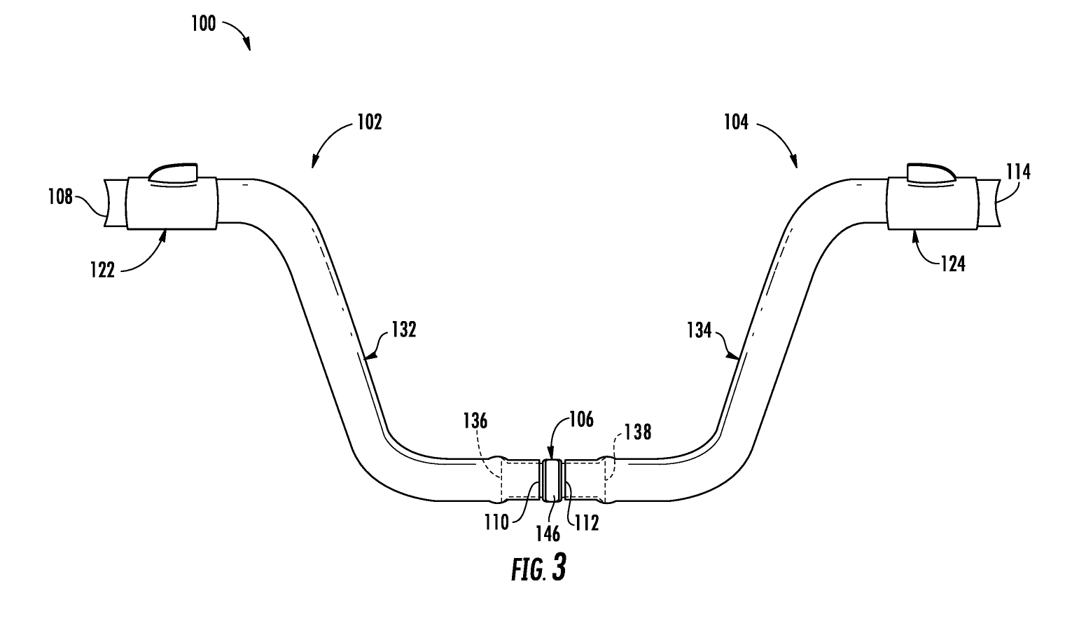

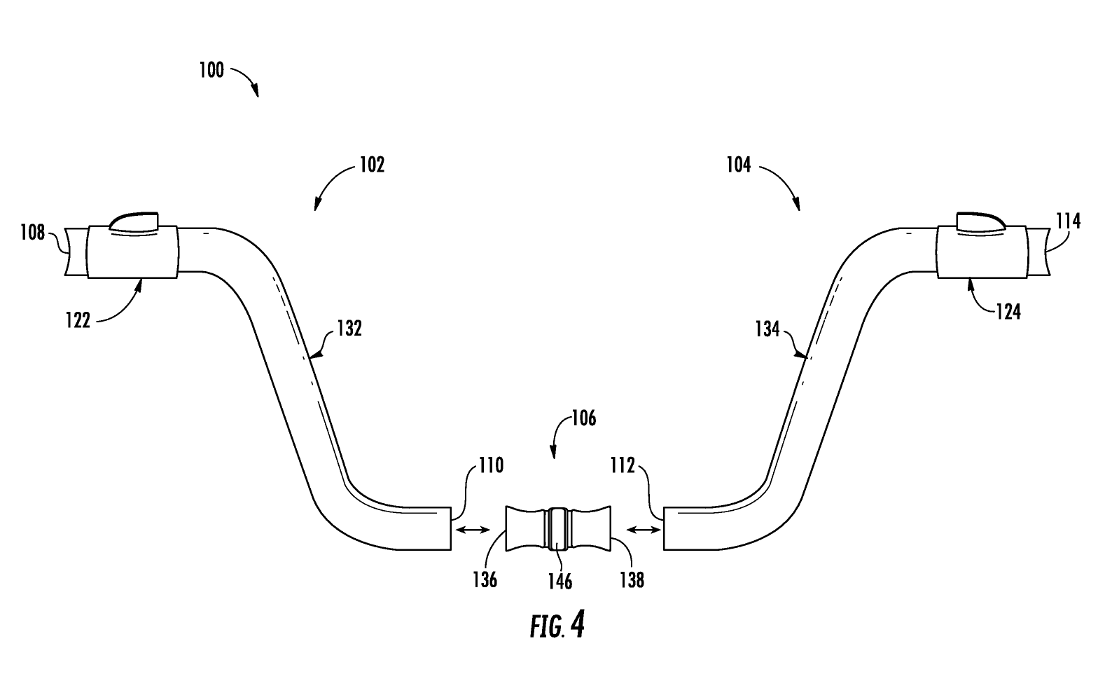

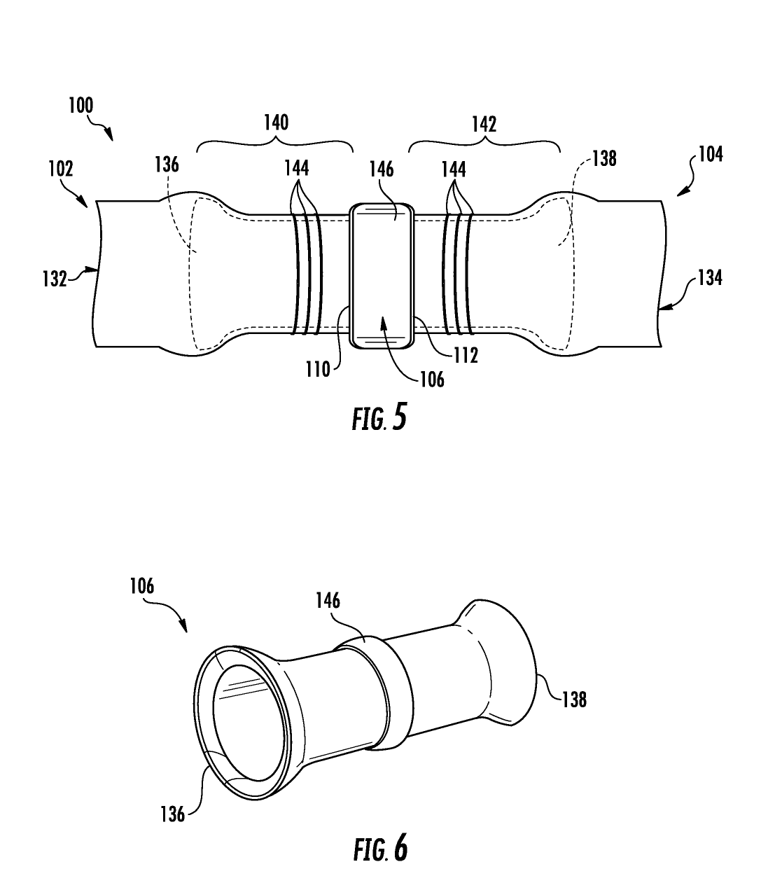

Referring now to FIGS. 3-6, differing views of the various system components described above with reference to FIG. 2 (less the tubing 128, 130 and the actuator assembly 126) are illustrated in accordance with aspects of the present subject matter. Specifically, FIGS. 3 and 4 illustrate assembled and unassembled views, respectively, of the valve tubes 102, 104 and the tube connector 106. FIG. 5 illustrates a close-up, assembled view of a portion of the components shown in FIG. 3, particularly illustrating the joint ends 110, 112 of the valve tubes 102, 104 coupled to the tube connector 106. Additionally, FIG. 6 illustrates a perspective view of the tube connector 106 shown in FIGS. 3-5.

As indicated above, the separate valve tubes 102, 104 of the disclosed system 100 may each include a valve device 122, 124 integrated therein and/or otherwise associated therewith. For instance, as shown in FIGS. 3 and 4, the first valve tube 102 includes a first valve device 122 positioned at or adjacent to its arterial end 108 while the second valve tube 104 includes a second valve device 124 positioned at or adjacent to its venous end 114. In addition, each valve tube 102, 104 may include a tube portion 132, 134 extending from its respective valve device 122, 124 and terminating at the opposed end of the valve tube 102, 104. For example, as shown in the illustrated embodiment, the first valve tube 102 may include a first tube portion 132 extending outwardly from the first valve device 122 to the first joint end 110 of the first valve tube 102. Similarly, the second valve tube 104 may include a second tube portion 134 extending outwardly from the second valve device 124 to the second joint end 112 of the second valve tube 104. In several embodiments, the tube portions 132, 134 of the valve tubes 102, 104 may be formed from a flexible, biocompatible material, such as expanded PTFE or any other suitable biocompatible tubing material.

It should be appreciated that, in several embodiments, the arterial/venous ends 108, 114 of the first and second valve tubes 102, 104 may be defined by their corresponding valve devices 122, 124 and/or their corresponding tube portions 132, 134. For instance, in one embodiment, the first tube portion 132 may be integrated into and/or extend through the first valve device 122 to the arterial end 108 of the first valve tube 102. Similarly, in one embodiment, the second tube portion 134 may be integrated into and/or extend through the second valve device 124 to the venous end 114 of the second valve tube 104.

As shown in the illustrated embodiment, the tube connector 106 may generally correspond to a hollow component extending lengthwise between a first end 136 and a second end 138, with the first end 136 being configured to be coupled to the first joint end 110 of the first valve tube 102 and the second end 138 being configured to be coupled to the second joint end 112 of the second valve tube 104. Specifically, as shown in FIGS. 3 and 5, the first joint end 110 of the first valve tube 102 may be configured to be positioned over the first end 136 of the tube connector 106 such that the first end 136 of the connector 106 is received within the first tube portion 132 of the first valve tube 102. Similarly, the second joint end 112 of the second valve tube 104 may be configured to be positioned over the second end 138 of the tube connector 106 such that the second end 138 of connector 106 is received within the second tube portion 134 of the second valve tube 104.

Such a configuration may create an overlapped joint or connection between the tube connector 106 and the valve tubes 102, 104. For example, as shown in FIG. 5, when the first end 136 of the tube connector 106 is received within the first tube portion 132, a first overlapped connection area 140 may be defined between the first valve tube 102 and the tube connector 106 that extends lengthwise between the first joint end 110 of the first valve tube 102 and the first end 136 of the tube connector 106. Similarly, when the second end 138 of the tube connector 106 is received within the second tube portion 134, a second overlapped connection area 142 may be defined between the second valve tube 104 and the tube connector 106 that extends lengthwise between the second joint end 112 of the second valve tube 104 and the second end 138 of the tube connector 106. The overlapped connection areas 140, 142 may provide locations for securely attaching the valve tubes 102, 104 to the tube connector 106. For instance, as shown in FIG. 5, one or more loop stiches 144 may be wrapped around the exterior of the valve tubes 102, 104 within the overlapped connection areas 140, 142 to couple the valve tubes 102, 104 to the tube connector 106. In another embodiment, the valve tubes 102, 104 may be coupled to the tube connector 106 at the overlapped connection areas 142, 144 using any other suitable means, such as by using an elastic band or ring that fits over the valve tubes 102, 14 within the overlapped connection areas 142, 144.

Additionally, as particularly shown in FIGS. 5 and 6, the tube connector 106 may include features or design elements that assist in coupling the valve tubes 102, 104 to the tube connector 106 and/or assist in positioning the joint ends 110, 112 of the valve tubes 102, 104 relative to the tube connector 106. For instance, as shown in the illustrated embodiment, the tube connector 106 may be flared outwardly at its first and second ends 136, 138. Such flared ends 136, 138 may serve as a retention means for preventing the valve tubes 102, 104 from being pulled off of the tube connector 106. For instance, when the loop stiches 144 are tightly wrapped around the valve tubes 102, 104 and the tube connector 106 within the respective overlapped connection areas 140, 142, the combination of the flared ends 136, 138 of the tube connector 106 and the loop stiches 144 may prevent the valve tubes 102, 104 from being removed from the tube connector 106. It should be appreciated that, as an alternative to the flared ends 136, 138, the tube connector 106 may include any other suitable means for retaining the valve tubes 102, 104 relative to the tube connector 106. For instance, in another embodiment, the tube connector 106 may define an annular lip or flange at each of its ends 136, 138 that is configured to interact with the loop stiches 144 to retain the valve tubes 102, 104 relative to the tube connector 106.

It should be appreciated that the ends 136, 138 of the tube connector 106 may also be rounded-off or non-sharp to avoid stress-risers and to prevent wear from occurring at the interface between the tube portions 132, 134 and the connector 106.

Additionally, as shown in FIG. 5, the tube connector 106 may include an annular rib 146 extending outwardly from a central portion of the tube connector 106 located between its first and second ends 136, 138. In general, the annular rib 146 may serve as a locating means for installing the valve tubes 102, 104 onto the tube connector 106. For instance, when installing the first valve tube 102 onto the tube connector 106, the first joint end 110 of the first valve tube 102 may be pushed inwardly relative to the tube connector 106 until the first joint end 110 contacts the annular rib 146. Similarly, when installing the second valve tube 104 onto the tube connector 106, the second joint end 112 of the second valve tube 104 may be pushed inwardly relative to the tube connector 106 until the second joint end 112 contacts the annular rib 146. As a result, the annular rib 146 may serve as a mechanical stop for the valve tubes 102, 104 and separate the joint ends 110, 112 of the valve tubes 102, 104. In addition, the annular rib 146 may also provide a visual indication to the user that the valve tubes 102, 104 have been properly installed onto the tube connector 106.

It should be appreciated that the tube connector 106 may generally be formed from any suitable material. For instance, in several embodiments, the tube connector 106 may be formed from a rigid, biocompatible material, such as titanium or a rigid, biocompatible polymer material.

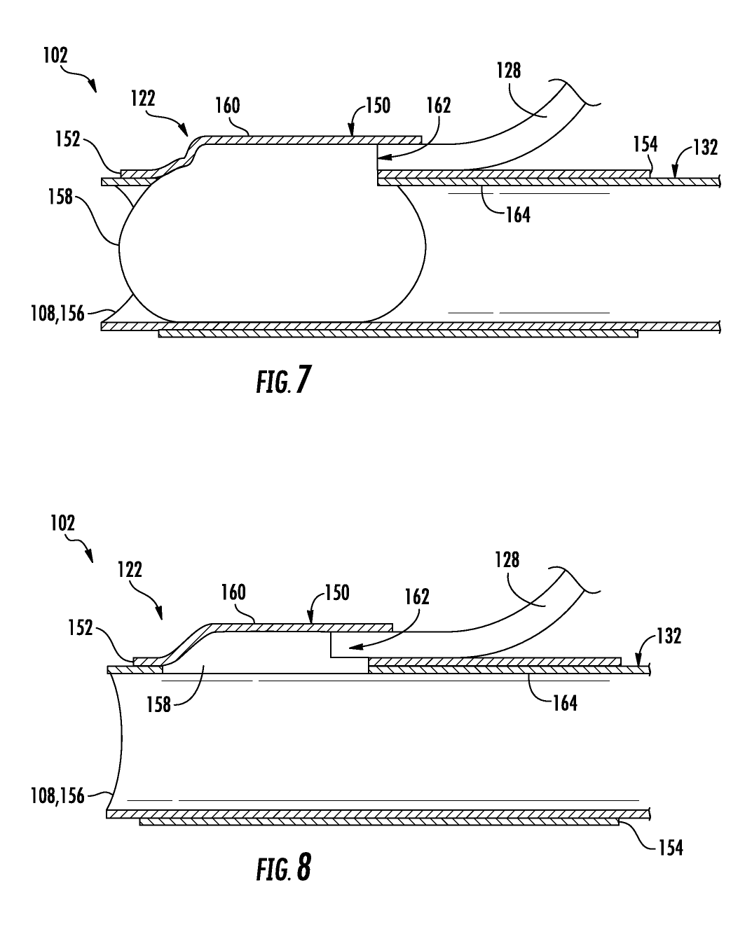

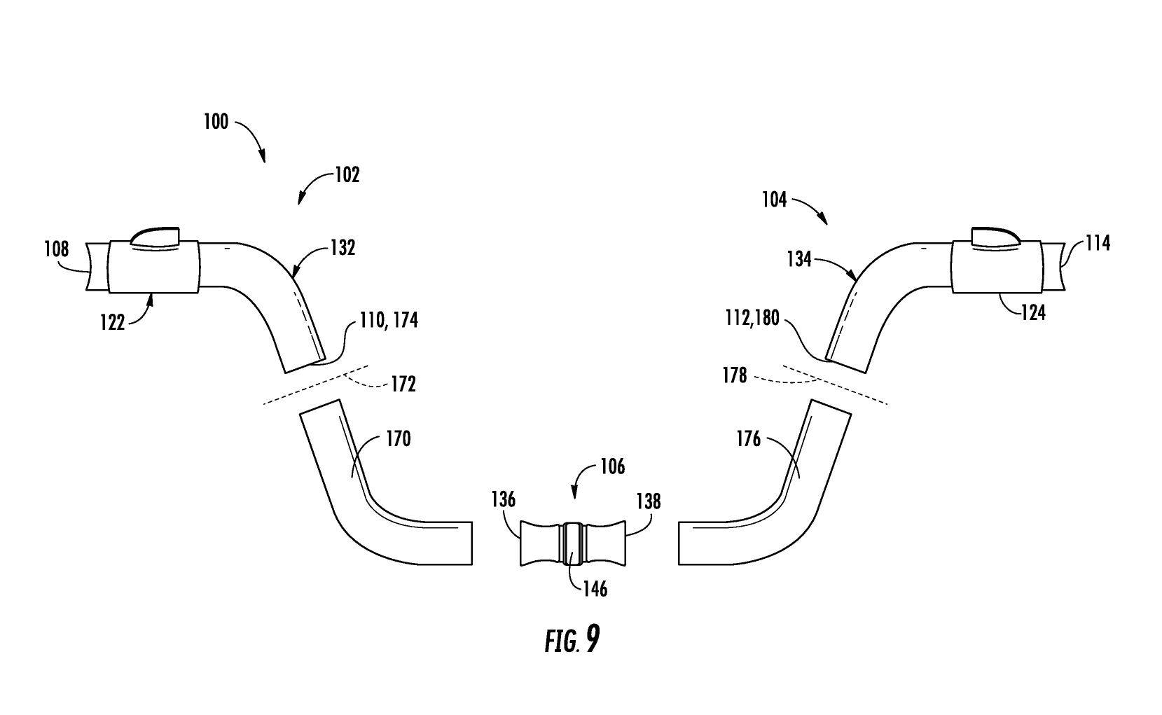

As indicated above, in several embodiments, each valve device 122, 124 may correspond to a balloon-actuated valve that is integrated into or otherwise disposed in-line within its corresponding valve tube 102, 104. For instance, FIGS. 7 and 8 illustrate one example of a suitable in-line balloon valve device that may be integrated into or otherwise associated with each valve tube 102, 104 of the disclosed system 100 in accordance with aspects of the present subject matter. Specifically, FIG. 7 illustrates the valve device in a closed position and FIG. 8 illustrates the valve device in an opened position. For purposes of discussion, the valve device shown in FIGS. 7 and 8 will be described as the first valve device 122 of the first valve tube 102. However, it should be appreciated that the second valve device 124 of the second valve tube 104 may be configured the same as or similar to the valve device shown in FIGS. 7 and 8.

As shown, the valve device 122 may include a housing or outer sleeve 150 extending lengthwise between a first end 152 disposed at or adjacent to the arterial end 108 of the first valve tube 102 and a second end 154 opposite the first end 152. In several embodiments, the sleeve 150 may be configured to extend around a section of the first tube portion 132 of the first valve tube 102. In such an embodiment, as shown in FIGS. 7 and 8, the first tube portion 132 may extend through the sleeve 150 such that an end 156 of the first tube portion 132 forms the arterial end 108 of the first valve tube 102, with the remainder of the tube portion 132 being configured to extend outwardly from the second end 154 of the valve device 122 to the first joint end 122 of the first valve tube 102. As such, the end 156 of the first tube portion 132 extending outwardly from the first end 152 of the sleeve 150 may be configured to be coupled to the artery 14 of the patient using any suitable means (e.g., sutures). Alternatively, the end 156 of the first tube portion 132 may be configured to be coupled to the second end 154 of the sleeve 150 using any suitable means (e.g., sutures). In such an embodiment, the first end 152 of the sleeve 150 may be configured to be coupled to the patient's artery 14 (e.g., using sutures).

Additionally, as shown in FIGS. 7 and 8, the valve device 122 may include a balloon 158 provided in operative association with the sleeve 150. For example, in several embodiments, the sleeve 150 may include an outward projection 160 configured to extend radially outwardly relative to the remainder of the sleeve 150 such that a recess 162 is defined directly below the outward projection 160 for receiving both the balloon 158 and the tubing 128 extending between the valve device 122 and the actuator assembly 126. As shown in FIG. 7, when fluid is supplied from the actuator assembly 126 to the balloon 158 via the tubing 128 so as to close the valve device 122, the balloon 158 may be configured to expand outwardly from the recess 162 into the interior of the sleeve 150 and/or first tube portion 132 such that the balloon 158 completely blocks the flow of blood through the first valve tube 102. Additionally, when the balloon 158 is deflated (as shown in FIG. 8), the balloon 158 may be retracted at least partially back into the recess 162 so that the balloon 158 provides no or minimal restriction to the flow of blood through the valve tube 102. For example, as shown in FIG. 8, in one embodiment, the balloon 158 may be configured to be retracted back into the recess 162 such that the inner surface of the balloon 158 is generally aligned within an inner surface 164 of the sleeve 150 and/or first tube portion 132.

As shown in FIGS. 7 and 8, the valve device 122 generally has a layered configuration, with the sleeve 150 serving as an outer layer and the first tube portion 132 serving as an inner layer through which the flow of blood passes. In such an embodiment, the sleeve 150 may be formed from a material having a sufficient hoop strength so as to provide a dimensional constraint to the balloon 158 as it is being inflated, thereby ensuring that the balloon 158 expands fully throughout the interior of the first tube portion 132 and completely blocks the flow of blood therethrough. Additionally, in one embodiment, an intermediate layer (not shown) may be provided between the sleeve 150 and the first tube portion 132. For instance, the intermediate layer may be formed from silicon, urethane or any other suitable material that serves as a “tie” or attachment layer between the sleeve 150 and the first tube portion 132.

It should be appreciated that the hoop strength of the outer sleeve 150 may generally correspond to any suitable value that allows the sleeve 150 to function as described herein. However, in a particular embodiment of the present subject matter, the outer sleeve 150 may have a hoop strength of greater than about 3,000 psi (e.g., 20.7 Mpa).

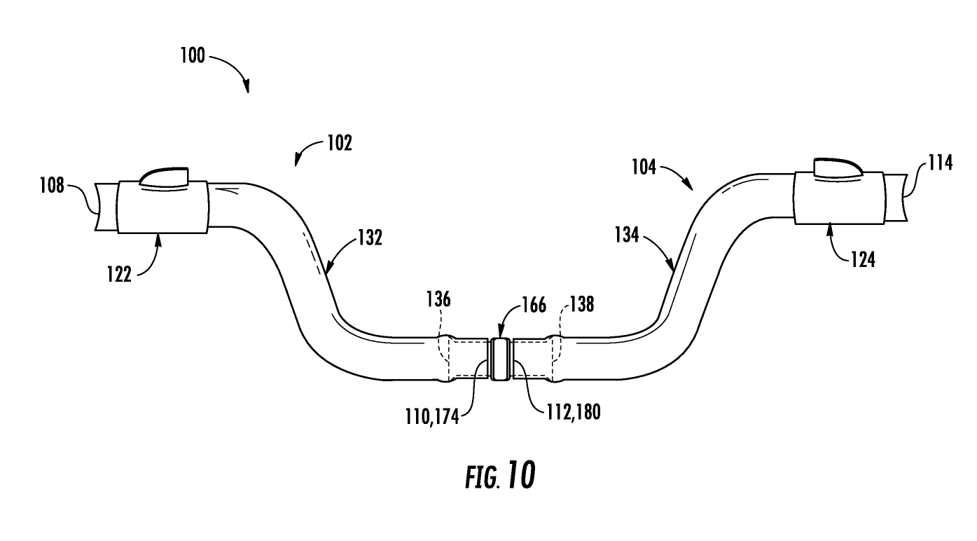

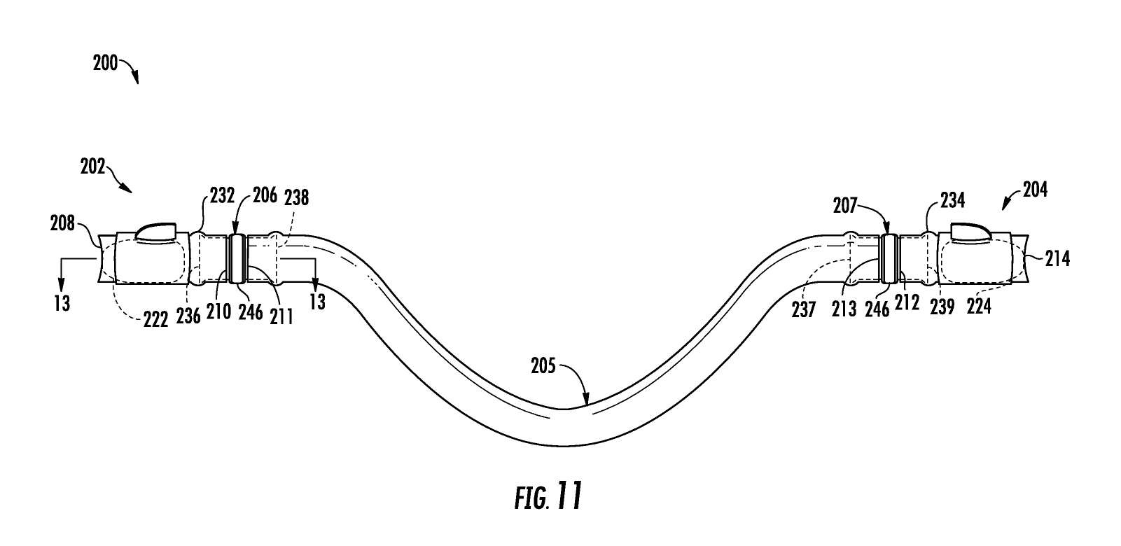

As indicated above, by providing an arteriovenous access valve system 100 including separate valve tubes 102, 104, the length of such valve tubes 102, 104 may be adjusted, as is desired or necessary, to suit the patient's body size, vascular system, access type and/or access location (e.g., the patient's arm versus the patient's thigh). For example, FIGS. 9 and 10 illustrate similar views of the system components shown in FIGS. 3 and 4, with portions of the valve tubes 102, 104 being cut-off to adjust the overall length of the flowpath to be formed between the patient's artery 14 and vein 16. As shown in FIG. 9, a first end section 170 of the first tube portion 132 has been cut-off or otherwise removed from the first valve tube 102 (e.g., at a first cut line 172) to shorten length of the first tube portion 132. In such an embodiment, the cut end of the first valve tube 102 formed at the first cut line 172 (e.g., first cut end 174) may correspond to the first joint end 110 of the valve tube 102. Similarly, as shown in FIG. 9, a second end section 176 of the second tube portion 134 has been cut-off or otherwise removed from the second valve tube 104 (e.g., at a second cut line 178) to shorten length of the second tube portion 134. In such an embodiment, the cut end of the second valve tube 104 formed at the second cut line 178 (e.g., second cut end 180) may correspond to the second joint end 112 of the valve tube 104. Thus, as shown in FIG. 10, the shortened valve tubes 102, 104 may then be coupled to the tube connector 106 at their newly formed cut ends 174, 180, with the first cut end 174 of the first valve tube 102 being positioned over and/or coupled the tube connector 106 at its first end 136 and the second cut end 180 of the second valve tube 104 being positioned over and/or coupled to the tube connector 106 at its second end 138.

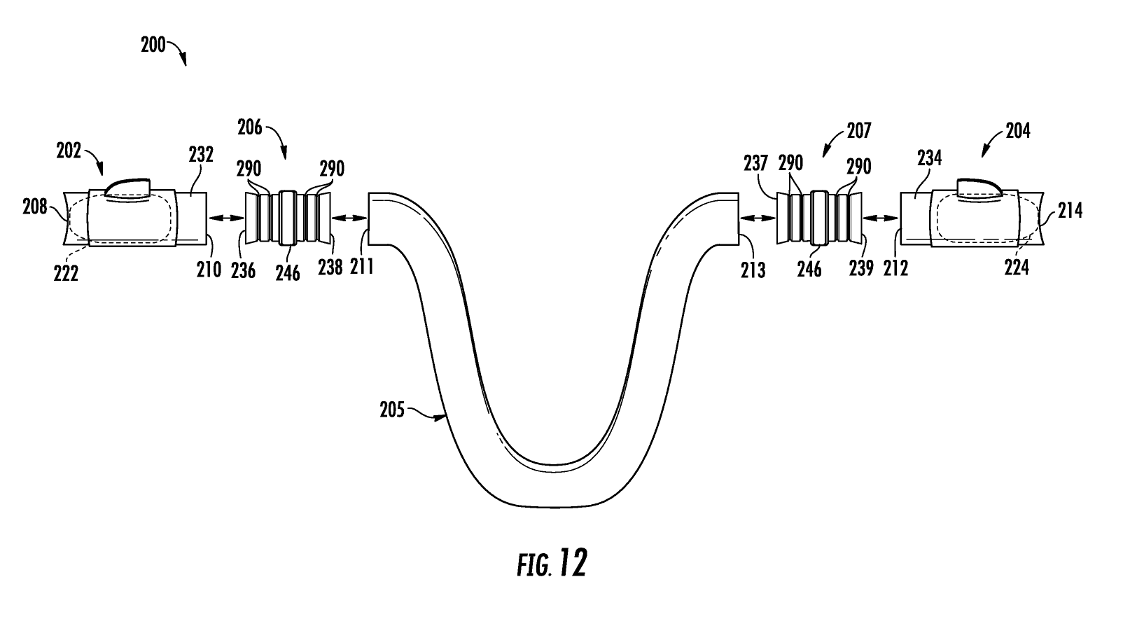

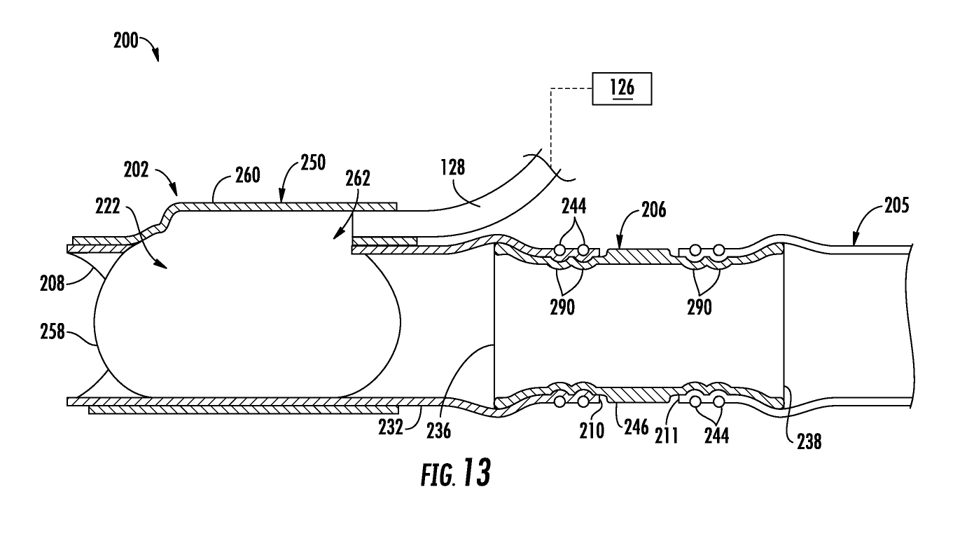

Referring now to FIGS. 11-13, another embodiment of an arteriovenous access valve system 200 is illustrated in accordance with aspects of the present subject matter. Specifically, FIGS. 11 and 12 illustrate assembled and unassembled views, respectively, of various components that may be included within the system 200. Additionally, FIG. 13 illustrates a cross-sectional view of a portion of the system components shown in FIG. 11 taken about line 13-13.

As particularly shown in FIGS. 11 and 12, the system 200 may include a first valve tube 202, a second valve tube 204, and an arteriovenous graft 205 configured to be coupled between the first and second valve tubes 202, 204 via first and second tube connectors 206, 207, respectively. In general, the first valve tube 202 may include an arterial end 208 configured to be coupled to the patient's artery 14 (FIG. 1) (e.g., via an anastomoses) and a first joint end 210 configured to be coupled to a corresponding first graft end 211 of the graft 205 via the first tube connector 206. Similarly, the second valve tube 204 may include a second joint end 212 configured to be coupled to a corresponding second graft end 213 of the graft 205 via the second tube connector 207 and a venous end 214 configured to be coupled to the patient's vein 16 (FIG. 1) (e.g., via an anastomoses). As such, when coupled together via the tube connectors 206, 207, the first and second valve tubes 202, 204 and the graft 205 may generally define a continuous flow path for blood extending between the patient's artery 14 and vein 16.

Similar to the valve tubes 102, 104 described above, the separate valve tubes 202, 204 of the disclosed system 200 may each include a valve device 222, 224 (indicated by dashed lines in FIGS. 11 and 12) integrated therein and/or otherwise associated therewith. For instance, as shown in FIGS. 11-13, the first valve tube 202 includes a first valve device 222 positioned at or adjacent to its arterial end 208 while the second valve tube 204 includes a second valve device 224 positioned at or adjacent to its venous end 214. In addition, each valve tube 202, 204 may include a tube portion 232, 234 extending between the respective ends of the valve tube. For example, as shown in the illustrated embodiment, the first valve tube 202 may include a first tube portion 232 extending between the arterial end 208 and the first joint end 210 of the first valve tube 202. Similarly, the second valve tube 204 may include a second tube portion 234 extending between the second joint end 212 and the venous end 214 of the second valve tube 204.

It should be appreciated that, in addition to the valve tubes 202, 204, the graft 205 and the tube connectors 206, 207, the system 200 may also include any other additional system components, such as the actuator assembly 126 and the tubing 128, 130 of the system 100 described above with reference to FIG. 2. For example, as shown in FIG. 13, each of the valve tubes 202, 204 may be coupled to the actuator assembly 126 via corresponding tubing 128, 130.

In several embodiments, the valve devices 222, 224 may correspond to balloon-actuated valves and, thus, may each include an inflatable balloon 258 (FIG. 13). Similar to the valve devices 122, 124 described above with reference to FIGS. 7 and 8, the balloon 258 may be provided in operative association with a housing or outer sleeve 250 within which the balloon 258 may be inflated/deflated. As particularly shown in FIG. 13, in one embodiment, the outer sleeve 250 of each valve device 222, 224 may be configured to extend around the outer perimeter of at least a portion of the tube portion 232, 234 of the corresponding valve tube 202, 204. In such an embodiment, the outer sleeve 250 may, for example, include an outward projection 260 configured to extend radially outwardly relative to the remainder of the sleeve 250 such that a recess 262 is defined directly below the outward projection 260 for receiving both the balloon 258 and the tubing 128 extending between the valve device 222 and the actuator assembly 126. As shown in FIG. 13, when fluid is supplied from the actuator assembly 126 to the balloon 258 via the tubing 128 so as to close the valve device 222, the balloon 258 may be configured to expand outwardly from the recess 262 into the interior of the first tube portion 232 such that the balloon 258 completely blocks the flow of blood through the first valve tube 202. Additionally, when the balloon 258 is deflated, the balloon 258 may be retracted at least partially back into the recess 262 so that the balloon 258 provides no or minimal restriction to the flow of blood through the valve tube 202.

It should be appreciated that the arteriovenous graft 205 may generally correspond to any suitable graft or other tube-like member. For example, the graft 205 may be formed from a flexible, biocompatible material, such as expanded PTFE or any other suitable biocompatible graft or tubing material. In another embodiment, the graft 205 may be formed from a woven or tissue-based material.

It should also be appreciated that the first and second tube connectors 206, 207 may generally be configured the same as or similar to the tube connector 106 described above. For example, the first tube connector 206 may generally correspond to a hollow component extending lengthwise between a first end 236 and a second end 238, with the first end 236 being configured to be coupled to the first joint end 210 of the first valve tube 202 and the second end 238 being configured to be coupled to the first graft end 211 of the graft 205. Specifically, as shown in FIGS. 11 and 13, the first joint end 210 of the first valve tube 202 may be configured to be positioned over the first end 236 of the first tube connector 206 such that the first end 236 of the connector 206 is received within a portion of the first valve tube 202. Similarly, the first graft end 211 of the graft 205 may be configured to be positioned over the second end 238 of the first tube connector 206 such that the second end 238 of the connector 206 is received within a portion of the graft 205.

Similarly, the second tube connector 207 may generally correspond to a hollow component extending lengthwise between a first end 237 and a second end 239, with the first end 237 being configured to be coupled to the second graft end 213 of the graft 205 and the second end 239 being configured to be coupled to the second joint end 212 of the second valve tube 204. Specifically, as shown in FIG. 11, the second graft end 213 of the graft 205 may be configured to be positioned over the first end 237 of the second tube connector 207 such that the first end 237 of the connector 207 is received within a portion of the graft 205. Similarly, the second joint end 212 of the second valve tube 204 may be configured to be positioned over the second end 239 of the second tube connector 209 such that the second end 239 of the connector 207 is received within a portion of the second valve tube 204.

Such a configuration may create overlapped joints or connections between the tube connectors 206, 207 and the both the valve tubes 202, 204 and the graft 205. Similar to the embodiment described above with reference to FIG. 5, the overlapped joints or connections may provide locations for securely attaching the ends of the valve tubes 202, 204 and the graft 205 to the tube connectors 206, 207. For instance, as shown in FIG. 13, one or more loop stiches 244 may be wrapped around the exterior of each valve tube 202, 204 and the graft 206 at the overlapped locations to couple each valve tube 202, 204 and each corresponding end 211, 213 of the graft 205 to their respective tube connector 206, 207. In another embodiment, the ends of the valve tubes 202, 204 and the graft 205 may be coupled to each tube connector 206, 207 using any other suitable means, such as by using an elastic band or ring that fits over the valve tubes 202, 204 and the graft 205 at the overlapped locations.

Additionally, similar to the tube connector 106 described above, each tube connector 206, 207 may also include features or design elements that assist in coupling the valve tubes 202, 204 and the graft 205 to the tube connector 206, 207 and/or assist in positioning the joint ends 210, 212 of the valve tubes 202, 204 and/or the ends 211, 213 of the graft 205 relative to the tube connector 206, 207. For instance, as shown in the illustrated embodiment, each tube connector 206, 207 may be flared outwardly at its first and second ends 236, 237, 238, 239 which may serve as a retention means for preventing the valve tubes 202, 204 and the graft 205 from being pulled off of the tube connector 206, 207. In addition, the ends 236, 237, 238, 239 of each tube connector 206, 206 may also be rounded-off or non-sharp. Moreover, as shown in the illustrated embodiment, each tube connector 206, 207 may also include an annular rib 246 extending outwardly from a central portion of the tube connector 206, 207 located between its first and second ends 236, 237, 238, 239 that may serve as a locating means for installing the valve tubes 202, 204 and the graft 205 onto the tube connectors 206, 207.

Further, in several embodiments, each tube connector 206, 207 may also include one or more inwardly extending channels or grooves 290 defined around its exterior. For example, as particularly shown in FIGS. 12 and 13, each tube connector 206, 207 may include one or more exterior grooves 290 located between the annular rib 246 and the first end 236, 237 of the connector 206, 207 and one or more exterior grooves 290 located between the annular rib 236 and the second end 238, 239 of the connector 206, 207. In one embodiment, the grooves 290 may generally be configured to receive or accommodate the attachment means used to couple the ends of the valve tubes 202, 204 and the graft 205 to each tube connector 206, 207. For instance, as shown in FIG. 13, the loop stiches 244 may be wrapped around the valve tubes 202, 204 and the graft 205 at the locations of the grooves 290 such that the stiches radially compress portions of the valve tube/graft into each groove 290. It should be appreciated that, in one embodiment, the tube connector 106 described above with reference to FIG. 6 may also be configured to include one or more inwardly extending grooves defined around its exterior.

It should be appreciated that, although the valve tubes 202, 204 shown in FIGS. 11 and 12 define lengths that are substantially shorter than the length of the graft 205, the valve tubes 202, 204 may define any other suitable lengths. For example, in one embodiment, the tube portions 232, 234 of the valve tubes 202, 204 may be elongated (e.g., similar to the embodiment shown in FIGS. 3 and 4) such that each valve tube 202, 204 defines a length that is equal to or greater than the length of the graft 205. Additionally, similar to the embodiment described above with reference to FIGS. 9 and 10, the lengths of the valve tubes 202, 204 and/or the length of the graft 205 may be adjustable, such as by cutting-off a portion of one or more of the valve tubes 202, 204 and/or the graft 205. Alternatively, multiple grafts 205 of varying lengths may be available for use within the system 200 such that the graft 205 defining the desired length may be selected for use with the valve tubes 202, 204.

It should also be appreciated that the present subject matter is also directed to a method for providing access between an artery and a vein of a patient. In one embodiment, the method may include assembling one or more of the components of the arteriovenous access valve system 100, 200 described herein (e.g., by coupling the joint ends 110, 112 of the valve tubes 102, 104 to the tube connector 106 or by coupling the valve tubes 202, 204 to the graft 205 via the first and second tube connectors 206, 207) and subsequently subcutaneously implanting such assembled components in the patient (e.g., by coupling the arterial end 108, 208 of the first valve tube 102, 202 to the patient's artery 14 and by coupling the venous end 114, 214 of the second valve tube 104, 204 to the patient's vein 16). In addition, the method may include opening first and second valve devices 122, 124, 222, 224 of the first and second valve tubes 102, 104, 202, 204 to allow blood to flow through the valve tubes 102, 104, 202, 204 between the artery 14 and the vein 16 of the patient and closing the first and second valve devices 122, 124, 222, 224 to prevent blood from flowing through the first and second valve tubes 102, 104, 202, 204 between the artery 14 and the vein 16 of the patient.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. An arteriovenous access valve system, the system comprising:
a first valve tube extending lengthwise between an arterial end and a first joint end, the first valve tube including a first valve device and a first tube portion extending outwardly from the first valve device towards the first joint end;
a separate second valve tube extending lengthwise between a venous end and a second joint end, the second valve tube including a second valve device and a second tube portion extending outwardly from the second valve device towards the second joint end; and
a tube connector extending lengthwise between a first end and a second end, the first joint end of the first valve tube being configured to be coupled to the tube connector at or adjacent to the first end, the second joint end of the second valve tube being configured to be coupled to the tube connector at or adjacent to the second end.

2. The system of claim 1, wherein the first and second valve devices each correspond to balloon-actuated devices, wherein, when a fluid is supplied to the first and second valve devices, each of the first and second valve devices is adjusted to a closed position and, when the fluid is drawn out of the first and second valve devices, each of the first and second valve devices is adjusted to an opened position.

3. The system of claim 2, wherein each of the first and second valve devices includes an inflatable balloon, wherein, when the fluid is supplied to the first and second valve devices, the inflatable balloon is inflated so as to adjust each of the first and second valve devices to the closed position and, when the fluid is drawn out of the first and second valve devices, the inflatable balloon is deflated so as to adjust each of the first and second valve device to the opened position.

4. The system of claim 2, further comprising an actuator assembly in fluid communication with the first and second valve devices, the actuator assembly being configured to both supply the fluid to each of the first and second valve devices so as to adjust the first and second valve devices to the closed position and draw the fluid out of each of the first and second valve devices so as to adjust the first and second valve devices to the opened position.

5. The system of claim 1, wherein the first joint end of the first valve tube is configured to be positioned over a portion of the tube connector such that the first end of the tube connector is received within the first tube portion and wherein the second joint end of the second valve tube is configured to be positioned over another portion of the tube connector such that the second end of the tube connector is received within the second tube portion.

6. The system of claim 5, wherein the first joint end of the first valve tube is received on the tube connector such that a first overlapped connection area is formed between the first valve tube and the tube connector and wherein the second joint end of the second valve tube is received on the tube connector such that a second overlapped connection area is formed between the second valve tube and the tube connector, wherein a stitched connection is formed between the first valve tube and the tube connector at the first overlapped connection area and between the second valve tube and the tube connector at the second overlapped connection area.

7. The system of claim 1, wherein the first and second ends of the tube connector are flared outwardly.

8. The system of claim 1, wherein the tube connector includes an annular rib extending outwardly therefrom between its first and second ends, the annular rib separating the first joint end of the first valve tube from the second joint end of the second valve tube when the first and second valve tubes are coupled to the tube connector.

9. The system of claim 1, wherein the tube connector includes at least one inwardly extending groove around an exterior of the tube connector.

10. The system of claim 1, wherein the arterial end of the first valve tube is configured to be coupled to an artery of a patient and the venous end of the second valve tube is configured to be coupled to a vein of the patient.

11. An arteriovenous access valve system, the system comprising:
a first valve tube extending lengthwise between an arterial end and a first joint end, the first valve tube including a first valve device;
a separate second valve tube extending lengthwise between a venous end and a second joint end, the second valve tube including a second valve device;
a graft extending lengthwise between a first graft end and a second graft end;
a first tube connector configured to couple the first joint end of the first valve tube to the first graft end of the graft; and
a second tube connector configured to couple the second end of the graft to the second joint end of the second valve tube.

12. The system of claim 11, wherein the first and second valve devices each correspond to balloon-actuated devices, wherein, when a fluid is supplied to the first and second valve devices, each of the first and second valve devices is adjusted to a closed position and, when the fluid is drawn out of the first and second valve devices, each of the first and second valve devices is adjusted to an opened position.

13. The system of claim 12, wherein each of the first and second valve devices includes an inflatable balloon, wherein, when the fluid is supplied to the first and second valve devices, the inflatable balloon is inflated so as to adjust each of the first and second valve devices to the closed position and, when the fluid is drawn out of the first and second valve devices, the inflatable balloon is deflated so as to adjust each of the first and second valve device to the opened position.

14. The system of claim 12, further comprising an actuator assembly in fluid communication with the first and second valve devices, the actuator assembly being configured to both supply the fluid to each of the first and second valve devices so as to adjust the first and second valve devices to the closed position and draw the fluid out of each of the first and second valve devices so as to adjust the first and second valve devices to the opened position.

15. The system of claim 11, wherein the arterial end of the first valve tube is configured to be coupled to an artery of a patient and the venous end of the second valve tube is configured to be coupled to a vein of the patient.

16. A method for providing access between an artery and a vein of a patient, the method comprising:
subcutaneously implanting an arteriovenous access valve system in the patient, the arteriovenous access valve system including a first valve tube extending lengthwise between an arterial end and a first joint end and a separate second valve tube extending lengthwise between a venous end and a second joint end, the first valve tube including a first valve device and the second valve tube including a second valve device, the arteriovenous access valve system further including at least one tube connector configured to be coupled to be coupled between the first and second valve tubes;
opening the first and second valve devices to allow blood to flow through the first and second valve tubes between the artery and the vein of the patient; and
closing the first and second valve devices to prevent blood from flowing through the first and second valve tubes between the artery and the vein of the patient.

17. The method of claim 16, wherein subcutaneously implanting the arteriovenous access valve system in the patient comprises coupling the arterial end of the first valve tube to the artery of the patient and coupling the venous end of the second valve tube to the vein of the patient.

18. The method of claim 16, further comprising coupling the at least one tube connector directly between the first and second valve tubes.

19. The method of claim 18, further comprising removing a section of at least one of the first valve tube or the second valve tube to adjust a length of the at least one of the first valve tube or the second valve tube prior to coupling the at least one tube connector directly between the first and second valve tubes.

20. The method of claim 16, wherein the at least one tube connector comprises a first tube connector and a second tube connector and wherein the arteriovenous access valve system further comprises a graft, further comprising coupling the first valve tube to an end of the graft via the first tube connector and coupling the second valve tube to an opposed end of the graft via the second tube connector.
